SEQUENCE LISTING (2 sequences)

SEQ ID NO: 1 - protein, 36 aa, synthetic construct
LFCKGGSCHFGGCPSHLIKVGSCFGFRSCCKWPWNA

SEQ ID NO: 2 - protein, 26 aa, synthetic construct
RVKRVWPLVIRTVIAGYNLYRAIKKK

(19) United States
(12) Patent Application Publication
(10) Pub. No.: US 2026/0102466 A1
(43) Pub. Date: Apr. 16, 2026

(54) ß-DEFENSIN-2 PEPTIDE FOR INHIBITING CELL GROWTH OF COLON CANCER

(71) Applicant: KING ABDULAZIZ UNIVERSITY, Jeddah (SA)

(72) Inventors: Mahmood RASOOL, Jeddah (SA); Absarul HAQUE, Jeddah (SA); Peter Natesan PUSHPARAJ, Jeddah (SA); Haitham A. YACOUB, Giza (EG); Maged Mostafa Mahmoud MOSTAFA, Jeddah (SA); Mohammed ALHARTHI, Jeddah (SA); Sajjad KARIM, Jeddah (SA); Adeel G. CHAUDHARY, Jeddah (SA)

(73) Assignee: KING ABDULAZIZ UNIVERSITY, Jeddah (SA)

(21) Appl. No.: 18/912,447

(22) Filed: Oct. 10, 2024

(51) Int. Cl. A61K 38/17; A61P 35/00

(52) U.S. Cl. CPC A61K 38/1729; A61P 35/00

(57) ABSTRACT

A method of treating colon cancer includes contacting the peptide chicken β-defensin-2 (AvBD-2) with a sample to treat colon cancer. The peptide inhibits the growth of cancer cells by downregulating PI3K and Akt1 signaling pathways.

### STATEMENT OF ACKNOWLEDGEMENT

This research work was funded by Institutional Fund Projects under grant no (IFPNC-013-141-2020). Therefore, authors gratefully acknowledge technical and financial support from the Ministry of Education and King Abdulaziz University, Jeddah, Saudi Arabia.

### REFERENCE TO SEQUENCE LISTING

In accordance with 37 CFR § 1.52 (e) (5) and with 37 CFR § 1.831, the specification makes reference to a Sequence Listing submitted electronically as a .xml file named “551147US Sequence Listing”. The .xml file was generated on Oct. 9, 2024, and is 2,481 bytes in size. The entire contents of the Sequence Listing are hereby incorporated by reference.

### BACKGROUND

### Technical Field

The present disclosure is directed to a method of treating colon cancer and, more particularly, towards the method of inhibiting cell growth of cancer cells in a sample using peptides, chicken β-defensin-2 (AvBD-2) and chicken cathelicidin-1 (CATH-1).

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Global increase in cancer due to genetic mutations that lead to uncontrolled cell growth is alarming. This rise is reflected in higher rates of both cancer diagnoses and deaths, emphasizing the need for effective solutions. Early detection and treatment options including surgery, chemotherapy, radiation, and targeted therapies such as immunotherapy are important to lowering cancer deaths. Colorectal cancer (CRC) is the third most commonly diagnosed cancer globally, and it has been shown to impose a large socio-economic burden. According to projections, the incidence of CRC is expected to increase by 60% by 2030, leading to an estimated 2.2 million new cases and 1.1 million deaths worldwide [García-Aranda, M. and Redondo, M., Immuntotherapy: A Chaalenge of Breast Cancer Treatment, Cancers (Basel), 2019, 11(12), 182]. These figures highlight the urgent need for advancements in diagnostics and treatments.

Despite modern diagnostic and treatment options, around 25% of patients are diagnosed with metastatic cancer, and nearly 40% develop metastasized tumors during the disease, often in the liver, spleen, lungs, or other organs. Oligometastasis, or single-site metastasis, impacts treatment and prognosis, allowing for more aggressive, tailored strategies that require careful monitoring and timely intervention for improved outcomes.

Chemotherapy has improved cancer treatment by targeting rapidly dividing cells; however, nonspecific toxicity is still a major issue affecting healthy cells. Recurrent tumors often develop because cancer cells become resistant to treatment, making long-term success difficult. Understanding resistance, using combination therapies, and developing new drugs that target resistant cells may help overcome problems of resistance and non-specificity. Traditional cancer treatments, including chemoradiotherapy and surgery, have been standard for improving survival; however, they face limitations in managing recurrent tumors, preventing side effects, and addressing multidrug resistance (MDR).

Investigating and validating targeted anticancer agents that may trigger cancer cell death by interfering with genes or proteins involved in cell proliferation or apoptosis resistance by targeting specific molecular alterations in DNA, RNA, and proteins that drive cancerous changes may assist in cancer survival rates [Pollack, L. A. et al., Introduction: charting the landscape of cancer survivors' health-related ourcomes and care, Cancer, 2009, 115, 4265-9].

Peptides are emerging as players in cancer treatment due to their ability to specifically target cancer cells, inhibit tumor growth, and induce cell death. Their small size allows them to penetrate cells and block cancer progression pathways; however, like many drugs, these peptides have drawbacks, and it is important to address these issues to enhance their efficacy and minimize adverse effects for clinical use. The low chemical and proteolytic stability, hemolytic activity, high cytotoxicity, and salt sensitivity of these peptides may be pharmacokinetic disadvantages that may hinder their clinical application [Kumar, S. D. et al., Cationic, amphipathic small molecules based on a triazine-piperazine-triazine scaffold as a new class of antimicrobial agents, Eur J Med Chem., 2022, 243, 114-747].

Antimicrobial peptides (AMPs), part of the innate immune system, have emerged as a therapeutic option for diseases, including cancer, since their discovery in 1939 and their isolation from animals in 1956. [Nguyen, T. T. T. et al., Avian antimicrobial peptides: in vitro and in ovo characterization and protection from early chick mortality caused by yolk sac infection, Sci Rep., 2021, 11(1), 21-32]. AMPs also have anti-inflammatory and immune-modulating properties, making them potential therapeutic agents for infections and other diseases, including cancer.

Molecular mechanisms underlying AMPs' ability to selectively modulate immune responses appear to be complex. It involves several signaling pathways (i.e., NF-κB), p38 and JNK mitogen-activated protein kinases, phosphoinositide 3-kinase (PI3K), and the engagement of various transcription factors. Intracellular uptake of peptides may or be mediated by G protein-coupled receptors. [Hancock, R. E. et al., the immunology of host defence peptides: beyond antimicrobial activity, Nat Rev Immunol, 2016, 16(5), 321-34]. Phosphoinositide 3-kinase (PI3K)/Akt is a signaling transduction pathway altered in cancer [Lawrence, M. S. et al., Discovery and saturation analysis of cancer genes across 21 tumour types, Nature, 2014, 23, 505, 74-84].

Although several cancer treatments have been developed in the past, more specific and resistant treatments are needed. An object of the present disclosure is directed to a method of treating colon cancer and, more particularly, towards the method of inhibiting cell growth of cancer cells in a sample using peptides, chicken β-defensin-2 (AvBD-2) and chicken cathelicidin-1 (CATH-1) to overcome drawbacks of the art. With their ability to target specific molecular pathways involved in cancer cell growth and survival, peptides like antimicrobial peptides (AMPs) may offer a more precise strategy to combat drug-resistant and aggressive tumors, marking steps forward in oncology.

### SUMMARY

In an exemplary embodiment, a method of treating colon cancer in a sample using a peptide is described. The method includes contacting the peptide, a chicken β-defensin-2 (AvBD-2) peptide, with a sample comprising colon cancer cells. The peptide inhibits cell growth of colon cancer cells through downregulation of a Phosphoinositide 3-Kinase (PI3K) signaling pathway and a RAC-alpha serine/threonine-protein kinase (Akt1) signaling pathway.

In some embodiments, the method includes the chicken β-defensin-2 peptide having an amino acid sequence at least 90% identical to SEQ ID No. 1.

In some embodiments, the method includes the chicken β-defensin-2 peptide having an amino acid sequence at least 95% identical to SEQ ID No. 1.

In some embodiments, the method includes the chicken β-defensin-2 peptide having an amino acid sequence at least 99% identical to SEQ ID No. 1.

In some embodiments, the chicken-defensin-2 peptide has a half-maximal inhibitory concentration (IC₅₀) of 865 to 875 μg/mL when administered to colorectal cancer cells, Human Colorectal Tumor 116 (HCT-116).

In some embodiments, the chicken β-defensin-2 peptide has a half-maximal inhibitory concentration of 565 to 575 μg/mL when administered to colorectal cancer cells, Human Tumor-29 (HT-29).

In some embodiments, the method includes contacting the chicken β-defensin-2 peptide at a concentration of 1000 μg/mL with colorectal cancer cells, HT-116, and reducing a cell viability of the colorectal cancer cells HCT-116 by 68 to 74%.

In some embodiments, the method includes contacting the chicken β-defensin-2 peptide at a concentration of 1000 μg/mL with colorectal cancer cells, HT-29, and reducing a cell viability of the colorectal cancer cells HT-29 by 97 to 99%.

In some embodiments, the method includes contacting colorectal cancer cells, HCT-116, with the chicken β-defensin-2 peptide and having a relative expression fold change of a PI3K gene of the colorectal cancer cells HCT-116 of 0.4 to 0.6 normalized to β-actin.

In some embodiments, the method includes contacting colorectal cancer cells, HCT-116, with the chicken β-defensin-2 peptide having a relative expression fold change of an Akt1 gene of the colorectal cancer cells HT-29 of 0.1 to 0.3 normalized to β-actin.

In some embodiments, the peptide is a chicken cathelicidin-1 (AvCath-1, AvCATH-1, and CATH-1) peptide.

In some embodiments, the method includes the chicken cathelicidin-1 peptide having an amino acid sequence at least 90% identical to SEQ ID No. 2.

In some embodiments, the method includes the chicken cathelicidin-1 peptide having an amino acid sequence at least 95% identical to SEQ ID No. 2.

In some embodiments, the method includes the chicken cathelicidin-1 peptide having an amino acid sequence at least 99% identical to SEQ ID No. 2.

In some embodiments, the chicken cathelicidin-1 peptide has a half-maximal inhibitory concentration of 64 to 68 μg/mL when administered to colorectal cancer cells, HCT-116.

In some embodiments, the chicken cathelicidin-1 peptide has a half-maximal inhibitory concentration of 74 to 78 μg/mL when administered to colorectal cancer cells, HCT-29.

In some embodiments, the method includes contacting the chicken cathelicidin-1 peptide at a concentration of 1000 μg/mL with colorectal cancer cells, HT-116, and reducing a cell viability of the colorectal cancer cells HCT-116 by 95 to 99%.

In some embodiments, the method includes contacting the chicken cathelicidin-1 peptide at a concentration of 1000 μg/mL with colorectal cancer cells, HT-29, and reducing a cell viability of the colorectal cancer cells HT-29 by 98.5 to 99.5%.

In some embodiments, the method includes contacting colorectal cancer cells, HCT-116, with the chicken cathelicidin-1 peptide and having a relative expression fold change of a PI3K gene of the colorectal cancer cells HCT-116 of 0.01 to 0.1 normalized to β-actin.

In some embodiments, the method includes contacting colorectal cancer cells, HCT-116, with the chicken cathelicidin-1 peptide having a relative expression fold change of an Akt1 gene of the colorectal cancer cells HT-29 of 0.05 to 0.3 normalized to B-actin.

In some embodiments, the method includes colorectal cancer cells, HT-29, having a cell viability of 0.5 to 1.5% after contacting the chicken cathelicidin-1 peptide at a concentration of 1000 μg/mL.

In some embodiments, the method includes colorectal cancer cells, HCT-116, contacted with the chicken cathelicidin-1 peptide having a relative expression fold change of a PI3K gene of 0.01 to 0.1 normalized to β-actin.

In some embodiments, the method includes colorectal cancer cells, HCT-116, contacted with the chicken cathelicidin-1 peptide having a relative expression fold change of a Akt1 gene of 0.05 to 0.3 normalized to B-actin.

These and other aspects of the non-limiting embodiments of the present disclosure will become apparent to those skilled in the art upon review of the following description of specific non-limiting embodiments of the disclosure in conjunction with the accompanying drawings. The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure (including alternatives and/or variations thereof) and many of the attendant advantages thereof will be readily obtained as the same becomes better understood by reference to the following detailed description when considered in connection with the accompanying drawings, wherein:

FIG. 1A is a graph depicting anticancer activity of cisplatin, illustrating the effect of varying cisplatin concentrations on the percent (%) cell viability against colon cancer cell lines Human Tumor-29 (HT-29) and Human Colorectal Tumor 116 (HCT-116), according to certain embodiments.

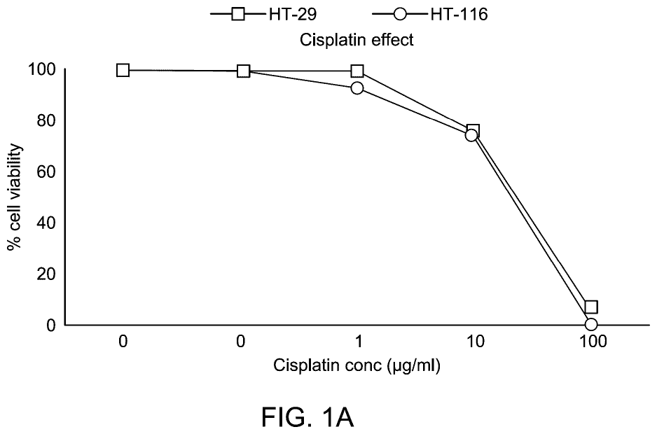

FIG. 1B is a graph depicting the effect of avian cathelicidin-1 (AvCath-1) and avian β-defensin-2 (AvBD-2) peptides on percent (%) cell viability against colon cancer cell lines HCT-116 cells, according to certain embodiments.

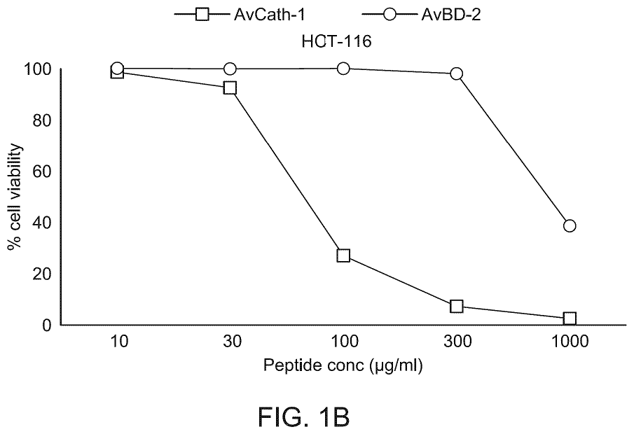

FIG. 1C is a graph depicting the effect of AvCath-1 and AvBD-2 peptides on percent (%) cell viability against colon cancer cell lines HT-29 cells, according to certain embodiments.

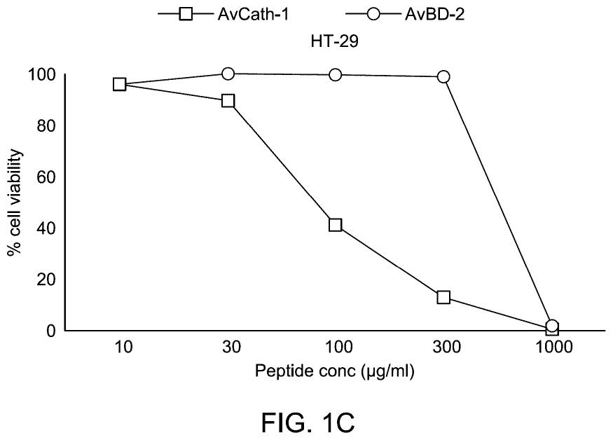

FIG. 2A is a graph illustrating a relative expression fold change of the PI3 kinase gene following treatment with cisplatin and AvCath-1 peptide in the CRC cell line HCT-116 and untreated cells (control), according to certain embodiments.

FIG. 2B is a graph illustrating the relative expression fold change of the Akt1 kinase B gene following treatment with cisplatin and AvCath-1 peptide in the CRC cell line HCT-116 and untreated cell (control), according to certain embodiments.

FIG. 3A is a graph illustrating the relative expression fold change of the PI3 kinase gene following treatment with cisplatin and AvBD-2 in the colon cancer cell line HCT-116 and untreated cells (control), according to certain embodiments.

FIG. 3B is a graph illustrating the relative expression fold change of kinase B (Akt1) gene following treatment with cisplatin and AvBD-2 in the colon cancer cell line HCT-116 and untreated cells (control), according to certain embodiments.

FIG. 4 depicts a western blot analysis depicting protein expression levels in PI3K signaling pathway and Akt1 signaling pathways in HCT-116 cells following treatment with cisplatin, AvBD-2, AvCath-1, and untreated cells as a control, according to certain embodiments.

### DETAILED DESCRIPTION

In the following description, it is understood that other embodiments may be utilized, and structural and operational changes may be made without departure from the scope of the present embodiments disclosed herein.

Reference will now be made to specific embodiments or features, examples of which are illustrated in the accompanying drawings. In the drawings, whenever possible, corresponding or similar reference numerals will be used to designate identical or corresponding parts throughout the several views. Moreover, references to various elements described herein are made collectively or individually when there may be more than one element of the same type. However, such references are merely exemplary in nature. It may be noted that any reference to elements in the singular may also be constructed to relate to the plural and vice-versa without limiting the scope of the disclosure to the exact number or type of such elements unless set forth explicitly in the appended claims.

When describing the present disclosure, the terms used are to be construed in accordance with the following definitions, unless a context dictates otherwise.

Embodiments of the present invention will now be described more fully hereinafter with reference to the accompanying drawings wherever applicable, in that some, but not all embodiments of the disclosure are shown.

In the drawings, reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “cancer” refers to all types of cancer, neoplasm or malignant tumors found in mammals (e.g. humans), including, but not limited to, leukemias, lymphomas, carcinomas, and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's disease, non-Hodgkin's lymphomas, and the like. Exemplary cancers that may be treated with a compound or method provided herein include cancer of the thyroid, endocrine system, brain, breast, cervix, colon, head & neck, liver, kidney, lung, ovary, pancreas, rectum, stomach, uterus, and the like. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, prostate cancer, combinations thereof, and the like.

An “anticancer agent” or “anticancer compound” as used herein refers to a molecule (e.g., compound, peptide, protein, nucleic acid, and the like) used to treat cancer through the destruction or inhibition of cancer cells or tissues. Anticancer agents may be selective for certain cancers or certain tissues.

“Anti-cancer agent” and “anticancer agent” or “anticancer compound” are used in accordance with their plain and ordinary meaning and refer to a composition (e.g. compound, drug, antagonist, inhibitor, modulator) having antineoplastic properties and/or the ability to inhibit the growth or proliferation of cells. In some embodiments, an anti-cancer agent is chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766, and the like), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan, and the like), ethylenimine and methylmelamines (e.g., hexamethlymelamine, thiotepa, and the like), alkyl sulfonates (e.g., busulfan and the like), nitrosoureas (e.g., carmustine, lomusitne, semustine, streptozocin, and the like), triazenes (decarbazine and the like)), anti-metabolites (e.g., 5-azathioprine, leucovorin, capecitabine, fludarabine, gemcitabine, pemetrexed, raltitrexed, folic acid analog (e.g., methotrexate and the like), or pyrimidine analogs (e.g., fluorouracil, floxouridine, Cytarabine, and the like), purine analogs (e.g., mercaptopurine, thioguanine, pentostatin, and the like), plant alkaloids (e.g., vincristine, vinblastine, vinorelbine, vindesine, podophyllotoxin, paclitaxel, docetaxel, and the like), topoisomerase inhibitors (e.g., irinotecan, topotecan, amsacrine, etoposide (VP16), etoposide phosphate, teniposide, and the like), antitumor antibiotics (e.g., doxorubicin, adriamycin, daunorubicin, epirubicin, actinomycin, bleomycin, mitomycin, mitoxantrone, plicamycin, and the like), platinum-based compounds (e.g., cisplatin, oxaloplatin, carboplatin, and the like), anthracenedione (e.g., mitoxantrone and the like), substituted urea (e.g., hydroxyurea and the like), methyl hydrazine derivative (e.g., procarbazine and the like), adrenocortical suppressant (e.g., mitotane, aminoglutethimide), epipodophyllotoxins (e.g., etoposide), antibiotics (e.g., daunorubicin, doxorubicin, bleomycin, and the like), enzymes (e.g., L-asparaginase and the like), inhibitors of mitogen-activated protein kinase signaling (e.g., U0126, PD98059, PD184352, PD0325901, ARRY-142886, SB239063, SP600125, BAY 43-9006, wortmannin, LY294002, and the like), Syk inhibitors, mTOR inhibitors, antibodies (e.g., rituxan and the like), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chlorins, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, 9-dioxamycin, diphenyl spiromustine, docosanol, dolasetron, doxifluridine, droloxifene, dronabinol, duocarmycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, 4, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, 06-benzylguanine, octreotide, okicenone, oligonucleotides, onapristone, ondansetron, ondansetron, oracin, oral cytokine inducer, ormaplatin, osaterone, oxaliplatin, oxaunomycin, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complexes, platinum compounds, platinum-triamine complexes, porfimer sodium, porfiromycin, prednisone, propyl bis-acridone, prostaglandin J2, proteasome inhibitors, protein A-based immune modulator, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylerie conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen-binding protein, sizofuran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitors, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, tazarotene, tecogalan sodium, tegafur, tellurapyrylium, telomerase inhibitors, temoporfin, temozolomide, teniposide, tetrachlorodecaoxide, tetrazomine, thaliblastine, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factors, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, zinostatin, stimalamer, adriamycin, dactinomycin, bleomycin, vinblastine, cisplatin, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cladribine, crisnatol mesylate, cyclophosphamide, cytarabine, dacarbazine, daunorubicin hydrochloride, decitabine, dexormaplatin, dezaguanine, dezaguanine mesylate, diaziquone, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, iimofosine, interleukin I1 (including recombinant interleukin II, or rlL.sub.2), interferon alfa-2a, interferon alfa-2b, interferon alfa-n1, interferon alfa-n3, interferon beta-la, interferon gamma-1b, iproplatin, irinotecan hydrochloride, lanreotide acetate, letrozole, leuprolide acetate, liarozole hydrochloride, lometrexol sodium, lomustine; losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazoie, nogalamycin, ormaplatin, oxisuran, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride, agents that arrest cells in the G2-M phases and/or modulate the formation or stability of microtubules, (e.g., Taxol™ (i.e. paclitaxel), Taxotere™, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e., WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39.HCl), AC-7700 (Ajinomoto, i.e., AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e. T-67, TL-138067 and TI-138067), COBRA-1 (Parker Hughes Institute, i.e., DDE-261 and WHI-261), H10 (Kansas State University), H16 (Kansas State University), Oncocidin Al (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, Inanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, and the like), monoclonal antibodies (e.g., anti-CD20, anti-HER2, anti-CD52, anti-HLA-DR, anti-VEGF monoclonal antibodies, and the like), immunotoxins (e.g., anti-CD33 monoclonal antibody-calicheamicin conjugate, anti-CD22 monoclonal antibody-pseudomonas exotoxin conjugate, and the like), immunotherapy (e.g., cellular immunotherapy, antibody therapy, cytokine therapy, combination immunotherapy, and the like), radioimmunotherapy (e.g., anti-CD20 monoclonal antibody conjugated to 111 In, 90Y, or 1311, and the like), immune checkpoint inhibitors (e.g., CTLA4 blockade, PD-1 inhibitors, PD-L1 inhibitors, and the like), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™)), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, and the like.

The terms “peptide,” “polypeptide,” and “protein,” as used herein, refer to a chain of amino acid residues linked by peptide (amide) bonds, regardless of the number of residues of amino acid constituting this chain. These terms can describe proteins, peptides, or polypeptides of any size, structure, or function. A protein, peptide, or polypeptide can exist as a single molecule or a complex of multiple molecules. Additionally, one or more amino acids within a protein, peptide, or polypeptide may be modified with chemical entities (e.g., carbohydrates, hydroxyl, phosphate, farnesyl, isofarnesyl groups, fatty acid groups, linkers, and the like) for purposes of conjugation, functionalization, and/or other modifications. Proteins, peptides, or polypeptides can be naturally occurring, recombinant, synthetic, any combination thereof, and the like. They may also be simple fragments of naturally occurring proteins or peptides. A peptide may comprise natural or non-natural amino acids.

As used herein, the term “amino acid” refers to the 20 natural standard amino acid residues (G, P, A, V, L, I, M, C, F, Y, W, H, K, R, Q, N, E, D, S, and T). These amino acids include A (Ala, alanine), R (Arg, arginine), N (Asn, asparagine), D (Asp, aspartic acid), C (Cys, cysteine), Q (Gln, glutamine), E (Glu, glutamic acid), G (Gly, glycine), H (His, histidine), I (Ile, isoleucine), L (Leu, leucine), K (Lys, lysine), M (Met, methionine), F (Phe, phenylalanine), P (Pro, proline), S (Ser, serine), T (Thr, threonine), W (Trp, tryptophan), Y (Tyr, tyrosine), and V (Val, valine). These amino acids combine in specific sequences to form peptides, which dictate the structure and function of proteins.

The term “therapeutic” refers to a procedure having to do with treating disease and helping healing take place in a subject with early or established signs of disease.

The term “treatment,” as used herein, refers to any intervention that alleviates, reduces, or improves a sign or symptom of a disease or pathological condition. In the context of the present invention, in the treatment of colorectal cancer (CRC), a chicken β-defensin-2 (AvBD-2) peptide and a cathelicidin-1 (CATH-1) peptide, with their specific amino acid sequences, disrupt cancer cell growth and tumor progression, thereby contributing to tumor destruction. The effectiveness of such treatments can be evaluated by measuring reductions in tumor size and/or the presence of viral peptides in treated tissue samples, such as those from the lungs or spleen. Treatment efficacy is determined by a measurable reduction in tumor or disease markers, ideally ranging from at least 10%, at least 20%, at least 30%, at least 40%, or at least 50% improvement, and more preferably at least 70%, at least 75%, at least 80%, at least 85%, at least 90%, at least 95%, at least 99%, or even complete (e.g., 99.5%, 99.8%, 99.9%, 100%) remission or suppression of disease. Successful treatment may be further demonstrated by reduced cell growth and/or enhanced inhibition of cancer cells before and after the intervention. In reference to disease management, the terms “treatment,” “treating,” and “therapy” also encompass any observable beneficial effects, such as delaying the onset of symptoms, reducing their severity, slowing disease progression, decreasing the frequency of recurrence, and/or improving the patient's overall health and well-being. These outcomes can be monitored through clinical parameters well-known in the field.

As used herein, the term “colorectal cancer cells” refers to cells originating from the epithelial tissue of the colon or rectum. These cells can undergo a series of genetic and epigenetic alterations, including mutations in genes such as APC, KRAS, TP53, and BRAF, which drive uncontrolled proliferation and invasive behavior. The development of colorectal cancer is often marked by either chromosomal instability (CIN) and/or microsatellite instability (MSI), contributing to the progression and heterogeneity of the disease. Colorectal cancer cells are utilized in laboratory settings to investigate tumor biology, drug resistance, and the efficacy of treatment strategies. Human colorectal cancer cell lines, such as HT-29, HCT-116, SW480, Caco-2 and the like, may be used to model disease characteristics and test potential therapies.

As used herein, the term “chicken β-defensin-2 peptide” refers to chicken β-defensin-2 (AvBD2), a peptide belonging to the β-defensin family, known for its antimicrobial properties. Chickens produce it as part of their innate immune response. β-defensins are small, cysteine-rich peptides that exhibit broad-spectrum antimicrobial activity against bacteria, viruses, and fungi. AvBD2 is effective in protecting against pathogens by disrupting microbial cell membranes and modulating the immune response. The peptide is synthesized in response to infection or inflammation and plays a role in the chicken's defense mechanisms.

As used herein, the term “chicken cathelicidin-1 peptide” refers to chicken cathelicidin-1 (AvCath-1, AvCATH-1, CATH-1, and Cath-1), an antimicrobial peptide belonging to the cathelicidin family. This peptide plays a role in chickens' innate immune response and defends against various pathogens, including bacteria, viruses, and fungi.

As used herein, the term “inhibitor” refers to a molecule or compound that interferes with the activity of a target, such as an enzyme, receptor, or signaling pathway, to reduce or halt its function. Inhibitors are used in various therapeutic applications, particularly in developing drugs for treating cancer, infectious diseases, and metabolic disorders. By binding to the active site or allosteric site of a target, inhibitors can prevent normal biological processes, such as blocking the replication of cancer cells, reducing inflammation, and/or stopping or slowing viral replication.

As used herein, the term “cell viability” refers to a measure of a cell's ability to maintain functional and structural integrity and continue to grow and divide. Evaluating cell viability involves determining the proportion of live cells within a population following exposure to potentially damaging treatments or conditions. Several methods are employed to assess cell viability. The Trypan Blue exclusion assay uses Trypan Blue dye to distinguish live cells from dead ones, as the dye penetrates only dead cells. The MTT assay measures mitochondrial enzyme activity by reducing a yellow dye to a purple formazan product, with the amount of formazan indicating viable cells. The sulforhodamine B (SRB) assay stains cellular proteins and quantifies the dye to reflect cell density and viability. The live/dead assay utilizes fluorescent dyes to differentiate between live cells with intact membranes and dead cells with compromised membranes. The XTT Assay uses a soluble dye to quantify cell viability spectrophotometrically, while the cell counting kit-8 (CCK-8) assay measures cell viability based on the reduction of a tetrazolium salt. These assays are used for evaluating treatment effectiveness, understanding cellular responses, and assessing the health and functionality of cell cultures in both research and clinical applications.

As used herein, “relative expression” refers to measuring gene or protein expression levels in a sample relative to a reference or control. This method is used in molecular biology to compare the expression of a target gene or protein under different conditions, such as in treated versus untreated cells or in diseased versus healthy tissues. Relative expression is often quantified using techniques like quantitative PCR (qPCR) for gene expression or Western blotting for protein expression, where the results are normalized against housekeeping genes or proteins that are consistently expressed across all samples. This normalization ensures that variations in sample quantity or quality do not affect accuracy of measurements. Relative expression is used for understanding biological effects of treatments, environmental conditions, and/or genetic alterations, providing insight into cellular function and disease mechanisms.

As used herein, the term “fold change” refers to a method to describe a relative difference in expression levels of a gene, protein, and/or other measurable entities between two experimental conditions. The value represents how often a value has increased or decreased. It is often used in gene expression studies and/or other biological assays to compare control and experimental groups. A fold change greater than 1 indicates upregulation (an increase in expression), while a fold change less than 1 indicates downregulation (a decrease in expression). For example, a fold change of 2 means the expression has doubled, while a fold change of 0.5 means it has halved. Fold change is used in conjunction with statistical significance tests to assess biological relevance in studies like RNA sequencing (RNA-seq), microarrays, and proteomics.

As used herein, the term “signaling pathway” refers to a series of molecular interactions that allow cells to respond to external stimuli and communicate with their environment. These pathways regulate cellular processes such as growth, differentiation, metabolism, and apoptosis. Typically, signaling pathways begin when a ligand, such as a hormone or growth factor, binds to a receptor on the cell surface. This binding activates a cascade of intracellular signaling proteins, including kinases, second messengers, and transcription factors, leading to specific cellular responses. Signaling pathways include the MAPK/ERK pathway, which is involved in cell growth and survival; the PI3K/AKT pathway, which regulates metabolism and apoptosis; and the Wnt/β-catenin pathway, which is involved in development and cancer. Dysregulation of signaling pathways is associated with diseases such as cancer, diabetes, and autoimmune disorders, making them a focus of targeted drug development. Understanding these pathways is used in developing therapies that can modulate cellular functions and treat diseases at the molecular level.

As used herein, the term “downregulation” refers to a process by which a cell decreases the quantity of a cellular component, such as a protein or a receptor, in response to external signals or environmental changes. This can occur at various levels, including gene expression, protein synthesis, and/or receptor availability on the cell surface. In the context of gene expression, downregulation may result in reduced transcription of specific genes, leading to lower amounts of corresponding proteins. In cellular signaling, downregulation may occur when a receptor is internalized or degraded after being activated by its ligand, reducing the cell's sensitivity to further stimulation. Downregulation is used in maintaining homeostasis and controlling cellular responses to stimuli. For example, downregulating oncogenes and/or other harmful proteins is a strategy to inhibit tumor growth in cancer treatments. Similarly, chronic exposure to a drug and/or hormone can lead to receptor downregulation, decreasing the cell's responsiveness over time, which is a mechanism underlying drug tolerance.

As used herein, the term “gene” refers to gene regulatory sequences (e.g., promoters, enhancers, and the like) and/or intron sequences. It will further be appreciated that the definition of a gene includes reference to a nucleic acid that does not encode a protein but rather encodes a functional RNA molecule (e.g., an RNAi agent, a ribozyme, a tRNA, and the like). It should be noted that for purposes of clarity, as used herein, the term “gene” generally refers to the portion of a nucleic acid that encodes a protein. The term may optionally include control sequences, which will be apparent to a person skilled in the art. This definition does not preclude the application of the term “gene” to expression units that do not encode the protein, but rather, in most cases, refers to the nucleic acid that encodes the protein when used herein.

According to an aspect of the present disclosure, a method of treating cancer is described. As used herein, “cancer” refers to human cancers, carcinomas, sarcomas, adenocarcinomas, lymphomas, leukemias, and the like, including, but not limited to, solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell and Large Cell lymphomas, and the like), Hodgkin's lymphoma, leukemia (including AML, ALL, CML, and the like), and/or multiple myeloma. In a preferred embodiment, the cancer is colon cancer.

The method includes treating colon cancer cells by contacting a sample with a peptide. One or more colon cancer cells are selected from the colon and/or rectum. Alternatively, the one or more colon cancer cell may be selected from the liver, lungs, lymph nodes, peritoneum, bones, and brain. The colon cancer cells may originate in the large intestine and, in advanced stages, spread to other areas of the body, such as the liver or lungs, and in rarer cases, to the bones and brain. The selection of colon cancer cells highlights the diverse locations within the body where colon cancer can metastasize, emphasizing the need for comprehensive treatment approaches. In some embodiments, the cancer cells are human colorectal cancer cell lines, including Human Colorectal Carcinoma Cell Line 116 (HCT-116), Human Colorectal Adenocarcinoma Cell Line 29 (HT-29), Sloan-Kettering Human Colorectal Adenocarcinoma Cell Line 480 (SW480), and the like, each representing different genetic backgrounds and stages of colorectal cancer progression. In a preferred embodiment, the colorectal cancer cell lines are HCT-116 and HT-29.

As used herein, the term “sample” or “biological sample” refers to a biological sample obtained from a subject, such as a fluid of a cell, tissue sample, and the like. In some cases, biological samples include genomic DNA, RNA (including mRNA and microRNA), proteins, combinations thereof, and the like. Examples of samples include saliva, blood, serum, urine, spinal fluid, tissue biopsy, surgical specimens, cells (e.g., PBMCs, white blood cells, lymphocytes, other immune system cells, and the like), and test materials. The peptide is a chicken β-defensin-2 (AvBD-2). Contacting the peptide with the sample inhibits cancer cell growth through downregulation of a phosphoinositide 3-kinase (PI3K) signaling pathway and an RAC-alpha serine/threonine-protein kinase (Akt1) signaling pathway.

In some embodiments, the peptide is a chicken cathelicidin-1 (AvCATH-1).

In some embodiments, peptides of specific lengths are selected from 10 to 100 amino acid residues, preferably 12 to 90 amino acid residues, preferably 14 to 80 amino acid residues, preferably 16 to 70 amino acid residues, preferably 18 to 60 amino acid residues, preferably 20 to 50 amino acid residues, more preferably 22 to 40 amino acid residues, and yet more preferably about 26 to 36 amino acid residues. In some embodiments, the amino acids are linked by peptide (amide) bonds, regardless of the number of amino acid residues constituting the chain. In some embodiments, amino acids can be selected based on their side chains, which may influence the protein's overall function. There are polar, non-polar, acidic, and basic amino acids, each contributing differently to the structure and reactivity of proteins. In an embodiment, the chicken β-defensin-2 peptide has an amino acid sequence at least 90%, preferably at least 91% preferably at least 92%, preferably at least 93%, preferably at least 94%, preferably at least 95%, preferably at least 96%, preferably at least 97%, preferably 98%, preferably at least 99%, more preferably at least 99.5%, and yet more preferably at least 99.9% identical to SEQ ID No. 1. SEQ ID No. 1 is LFCKGGSCHFGGCPSHLIKVGSCFGFRSCCKWPWNA and has a molecular weight (M.W.) of 3921.66. In an embodiment, the chicken cathelicidin-1 peptide has an amino acid sequence at least 90%, preferably at least 91% preferably at least 92%, preferably at least 93%, preferably at least 94%, preferably at least 95%, preferably at least 96%, preferably at least 97%, preferably 98%, preferably at least 99%, more preferably at least 99.5%, and yet more preferably at least 99.9% identical to SEQ ID No. 2. SEQ ID No. 2 is RVKRVWPLVIRTVIAGYNLYRAIKKK and has a molecular weight of 3399.92. This high degree of sequence similarity helps the peptides maintain their functional and structural integrity, which is for their effectiveness in therapeutic applications.

In a preferred embodiment, peptides are administered to/contacted with colorectal cancer cells in a sample to evaluate their efficacy in inhibiting cell growth. The term “administration” means either a direct administration of a compound or composition of the invention, or administration of a prodrug, derivative, and/or analog that forms an equivalent amount of active compound or substance in the body. Exemplary routes of administration include injection (subcutaneous, intramuscular, intradermal, intraperitoneal, intravenous, and the like), oral, sublingual, rectal, transdermal, intranasal, vaginal, inhalation routes, combinations thereof, and the like.

In some embodiments, the AvBD-2 has a half-maximal inhibitory concentration (IC₅₀) of 865 to 875 μg/mL, preferably 866 to 873 μg/mL, preferably 867 to 870 μg/mL, more preferably 868 to 869 μg/mL, and yet more preferably about 868.91 μg/mL for treating HCT-116 cells. In other embodiments, the AvBD-2 has a IC₅₀ of 565 to 575 μg/mL, preferably 567 to 574 μg/mL, preferably 567 to 573 μg/mL, preferably 568 to 572 μg/mL, more preferably 569 to 570 μg/mL, and yet more preferably about 569.31 μg/mL for treating HT-29 cells. In some embodiments, the chicken cathelicidin-1 peptide comprises a half-maximal inhibitory concentration (IC₅₀) of 64 to 68 μg/mL, preferably 65 to 67 μg/mL, more preferably 65.3 to 66 μg/mL, and yet more preferably about 65.58 μg/mL when administered to colorectal cancer cells, specifically HCT-116. In some other embodiments, the chicken cathelicidin-1 peptide exhibits a slightly higher IC₅₀, ranging from 74 to 78 μg/mL, preferably 75 to 77 μg/mL, more preferably 76 to 76.5 μg/mL, and yet more preferably about 76.11 μg/mL for colorectal cancer cells HT-29. In some embodiments, IC₅₀ values of the CATH-1 peptide are about 76.11 μg/mL and 65.58 μg/mL for HT-29 and HCT-116, respectively. In some embodiments, IC₅₀ values of the AvBD-2 peptide are about 569.31 μg/mL and 868.91 μg/mL for HT-29 and HCT-116, respectively. Both the cell lines exhibited sensitivity to AvBD-2 and CATH-1 peptides.

In certain embodiments, the method includes assessing the cell viability of chicken cathelicidin-2 and beta-defensin-2 peptides in drug-resistant colon cancer cell lines (HT-29 and HCT-116). The cell viability may be assessed by any of the methods known in the art—for example, Trypan Blue exclusion assay, MTT assay, sulforhodamine B (SRB) assay, live/dead assay, flow cytometry, and the like. In some embodiments, the colorectal cancer cells HT-116 exhibit a cell viability of 36 to 42%, preferably, 37 to 41%, preferably 38 to 40%, and preferably about 39% after exposure to the AvBD-2 peptide at a concentration of 1000 μg/mL. In other embodiments, colorectal cancer cells, HT-29, show a cell viability of 1 to 3%, preferably 1.5 to 2.5%, and preferably about 2% following treatment with the AvBD-2 peptide at a concentration of 1000 μg/mL. In some embodiments, the colorectal cancer cells HT-116 exhibit a cell viability of 1 to 5%, preferably, 2 to 4%, and preferably about 3% after exposure to the chicken cathelicidin-1 peptide at a concentration of 1000 μg/mL. In other embodiments, colorectal cancer cells, HT-29, show a cell viability of 0.5 to 1.5%, preferably 0.7 to 1.2%, and preferably about 1% following treatment with the chicken cathelicidin-1 peptide at a concentration of 1000 μg/mL. The results indicate that both peptides inhibitory effects on colorectal cancer cells with varying degrees of efficacy depending on the specific cell line.

In certain embodiments, the method includes assessing gene expression changes in colorectal cancer cells after treatment with the chicken β-defensin-2 peptide. The term “gene expression” refers to the process by which genetic information encoded in a gene is converted into functional products, such as proteins or RNA molecules. It occurs in two main stages: transcription, where the DNA sequence of a gene is transcribed into messenger RNA (mRNA), and translation, where the mRNA is translated into a protein. Cancer cells often exhibit upregulated (increased) or downregulated (decreased) expression of genes that regulate cell growth, division, and/or survival. In an embodiment, the relative expression fold change of the PI3K gene after treating colorectal cancer cells HCT-116 with the chicken β-defensin-2 peptide is 0.4 and 0.6, preferably 0.45 to 0.55, and preferably about 0.5, normalized to β-actin. In some embodiments, the relative expression fold change of the Akt1 gene after treating colorectal cancer cells, HCT-116 with the chicken β-defensin-2 peptide is 0.1 to 0.3, preferably 0.15 to 0.25, and preferably about 0.2, normalized to β-actin. Fold change refers to the ratio of gene expression levels in treated cells compared to control cells, providing insights into the impact of the peptide on specific signaling pathways. In some embodiments, colorectal cancer cells of the HCT-116 line treated with chicken cathelicidin-1 peptide exhibit a relative expression fold change in the PI3K gene ranging from 0.01 to 0.1, preferably 0.02 to 0.08, and preferably about 0.05, normalized to β-actin. In some embodiments, colorectal cancer cells of the HCT-116 line treated with chicken cathelicidin-1 peptide exhibit a relative expression fold change of Akt1 gene between 0.05 and 0.3, preferably 0.1 to 0.2, and preferably about 0.15, normalized to β-actin.

Aspects of the present disclosure provide a method for inhibiting the proliferation of cancer cells in a sample, i.e., a human body. As described, the method is efficient, economically sustainable, and easily employable.

### EXAMPLES

The following examples describe and demonstrate a method of treating colon cancer using chicken β-defensin-2 (AvBD-2) and chicken cathelicidin-1 (CATH-1) peptides by inhibiting cell growth through downregulation of a PI3K signaling pathway and an Akt1 signaling pathway as described herein. The examples are provided solely for illustration and are not to be construed as limitations of the present disclosure, as many variations thereof are possible without departing from the spirit and scope of the present disclosure.

### Example 1: Materials and Methods

The experimental procedures were carried out in triplicate and were repeated three times to minimize discrepancies and variability in the results. In order to assess the potential for false positives, negative controls were incorporated into all experimental setups. These controls allowed for the evaluation of chicken cathelicidin-1 and β-defensin-2 peptides' effects on cancer cell lines in their absence. The study protocol was approved by the Bio Medical Ethics Committee Board at King Abdulaziz University, with reference number 325-19.

Peptides: The synthesis of mature chicken cathelicidins was obtained from Peptide 2.0, United States (http://www.peptide2.com). The peptides were purified through HPLC, with a purity of up to 95%. Validity of the peptides was confirmed through mass spectrometry analysis. The amino acid sequences of the mature peptides are presented in Table 1.

TABLE 1
The amino acids sequence of the chicken
cathelicidin-1 and β-defensin-2 peptides

| Peptide | Amino Acid Sequence | M.W |
|---|---|---|
| Cathelicidin-1 | RVKRVWPLVIRTVIAGYNL | 3399.92 |
| | YRAIKKK | |
| β-defensin-2 | LFCKGGSCHFGGCPSHLIK | 3921.66 |
| | VGSCFGFRSCCKWPWNA | |

### Example 2: Cell Culture and Treatment

The human colonic cell line HCT-116 was obtained from the American Type Culture Collection (Manassas, VA, USA). The colorectal cell lines were maintained in Dulbecco's Modified Eagle Medium (DMEM) with high glucose supplemented with 10% FBS, 2.5 mM L-glutamine, 1.2 g/L sodium bicarbonate, 0.5 mM hydrocortisone, 400 ng/mL hydrocortisone, and 15 mM HEPES (4-(2-hydroxyethyl) piperazine-1-ethansulfonic acid). The cell culture flasks were incubated at 37° C. in a humidified incubator with 5% CO2 and 95% air, and the culture medium was replenished every 2-3 days to achieve 80-90% confluency after 5-7 days. The cells were then sub-cultured by trypsinization and seeded in fresh T75 flasks containing complete DMEM. After reaching 90% confluence, the cells were harvested and counted. The HT-29 and HCT-116 cells were seeded in triplicate 96 well plates at a density of 5×10³ cells/cm² and treated with chicken cathelicidin-1 and β-defensin-2 peptides for 48 hours incubation, as previously described [Liu, Y. et al., MicroRNA-126 functions as a tumor suppressor in colorectal cancer cells by targeting CXCR4 via the AKT and ERK1/2 signaling pathways, Int J Oncol., 2014, 44(1), 203-10, which is incorporated herein by reference in its entirety].

### Example 3: Cell Viability Assay

The sulforhodamine B (SRB) test was employed to evaluate altered cell viability in drug-resistant colon cancer cell lines (HT-29 and HCT-116) using chicken cathelicidin-2 and beta-defensin-2 peptides to assess their anticancer efficacy. A total of 100 μL of cell suspension (5×10³ cells) was seeded in 96-well plates with complete media. After 24 hours of incubation, the cells were treated with various drug concentrations formulated in 100 μL media. The culture medium was replaced after 72 hours of drug exposure, and the cells were fixed with 150 μL of 10% TCA and incubated at 4° C. for 60 minutes. Subsequently, the cells were washed five times with distilled water, and 70 μL SRB solution (0.4% w/v) was added to the plates and incubated in a dark place at room temperature for 10 minutes. The plates were washed three times with 1% acetic acid and kept overnight at room temperature for air drying. Then, 150 μL of TRIS (10 mM) was added to dissolve the protein-bound SRB stain, and the absorbance was recorded at 540 nm using a BMG LABTECH®-FLUOstar Omega microplate reader (Ortenberg, Germany). The median inhibitory concentration (IC₅₀) was calculated using the probability model. The inhibition rate of cell proliferation was calculated as: inhibition rate (%)=1−A₅₄₀ (test)/A₅₄₀ (control)×100%. The data were calculated and evaluated from three independent experiments, each performed in triplicate [Allam, R. M. et al., Fingolimod interrupts the cross talk between estrogen metabolism and sphingolipid metabolism within prostate cancer cells, Toxicol Lett., 2018, 291, 77-85, which is incorporated herein by reference in its entirety].

### Example 4: RNA Extraction, cDNA Synthesis, and qPCR Analysis

Extraction of total RNA from HCT-116 cells that had been treated with cisplatin, chicken cathelicidin-1, and β-defensin-2 was carried out using a GENEJET RNA purification kit (Invitrogen, USA). The concentration and purity of the RNA were subsequently determined using a NanoDrop 2000 (Thermo Fisher Scientific, USA). The first-strand complementary DNA (cDNA) from 1 μg of total RNA was synthesized using a first-strand cDNA synthesis kit (Promega, USA), according to the manufacturer's protocol. Briefly, the reverse transcriptase (RT) reaction mixture containing the PCR tube was subjected to thermal cycling conditions, which included an initial incubation at 25° C. for 5 minutes, followed by incubation at 42° C. for 60 minutes, and 70° C. for 15 minutes, and finally held at 4° C. The synthesized cDNA samples were immediately stored at −20° C. until further use. Quantitative polymerase chain reaction (qPCR) was performed using the SYBR® Green Master Mix (Qiagen, Germany), and the reaction was carried out in 48 well optical plates using a Real-Time PCR Detection System (Applied Biosystems, USA). The qPCR extension protocol followed the instructions in the SYBR® Green Master Mix manual (Qiagen, Germany). The following primers were utilized for qPCR: For the PI3K gene, the forward sequence was 5-GAAGCACCTGAATAGGCAAGTCG-3, and the reverse sequence was 5-GCTGGGTCTTCTCCTGTTCT-3. For the Akt1 gene, the forward sequence was 5-TGGACTACCTGCACTCGGAGAA-3, and the reverse sequence was 5-GTGCCGCAAGTCTTCATGG-3. The endogenous control primer pairs were β-actin, forward primer 5-CACCATTGGCAATGAGCGGT-3, and reverse primer 5-AGGTCTTTGCGGATGTCCAC-3, which were used to normalize the expression data. The expression of the genes of interest was determined by calculating the difference between the Ct value of the gene of interest and that of the endogenous control gene.

To assess comparative gene expression, the ΔΔCt value was calculated, which signifies the difference between the Ct values of the treated and control groups. The 2-ΔΔCt value was then utilized to represent the fold expression change in the gene of interest between the treated and control groups. A 2-ΔΔCt value greater than 1 indicates that gene expression in the treated groups was higher than in the control groups [Livak, K. J. and Schmittgen, T. D., Analysis of relative gene expression data using real-time quantitative PCR and the 2 (−Delta Delta C (T)) Method, Methods, 2001, 25(4), 402-8, which is incorporated herein by reference in its entirety].

### Example 5: Western Blotting for Determination of Protein Expression of PI3K/Akt

After administering cisplatin, chicken β-defensin-2, and cathelicidin-1 peptides to HCT116-cells for 24 hours, the cell culture plates were washed three times with PBS buffer, and the cells were detached using a 0.25% trypsin and 0.03% EDTA solution (Invitrogen Life Technologies). The extracted proteins were quantified and analyzed using a Nano Drop spectrophotometer. The proteins were then separated using SDS-PAGE (Bio-Rad, USA) and transferred to polyvinylidene fluoride (PVDF) membranes. The membranes were blocked with 5% BSA for two hours at room temperature and incubated with primary antibodies, including rabbit anti-human Akt1 (1:1000), PAkt1 (1:1000), PI3K (1:1000), purchased from Cell signaling Technology (Beverly, MA, USA), and GAPDH antibodies overnight at 4° C. The membranes were washed three times at five-minute intervals at room temperature, followed by treatment with secondary antibodies: horseradish peroxidase-conjugated anti-rabbit (1:3000, Invitrogen, USA) and anti-mouse IgG (1:3000, Cell signaling Technology, USA) antibodies for two hours. The membranes were then exposed to chemiluminescence substrate ECL for detection of the immunoblots through imaging. The Puregene (Genetix) protein ladder was used as a reference to determine the size of the immunoblotting proteins. The chemiluminescence reaction was performed using ECL western blot HRP substrate (Pierce, Thermo Fisher Scientific), and image acquisition was performed using the ChemiDoc imaging system (Bio-Rad). The intensities of the bands were calculated using the ImageJ software and normalized to the respective GAPDH band intensities. The results are represented as fold change with respect to the control.

### Example 6: Statistical Analysis

Statistical data presented in the present disclosure are expressed in terms of the mean±standard deviation. The data analysis was conducted using Microsoft Excel 2020, and statistical significance was determined using the student's t-test for comparisons between two groups and one-way ANOVA for multiple comparisons. A significance level of P<0.05 was employed for all statistical tests. To ensure the reliability of the results, experiments were repeated a minimum of three times. For data with error bars, the mean values and standard deviations from triplicate repetitions are shown.

The sulforhodamine B (SRB) assay was conducted to assess the cytotoxicity and anticancer efficacy of chicken cathelicidin-2 and β-defensin-2 peptides in drug-resistant colon cancer cell lines (HT-29 and HCT-116). Cisplatin, a well-known chemotherapeutic drug, was used as a positive control. Both cell lines were sensitive to cisplatin treatment at low concentrations, with IC₅₀ values of 19.35 μg/mL and 15.59 μg/mL for HT-29 and HCT-116, respectively. Moreover, HCT-116 cells were found to be less viable than HT-29 cells after treatment with cisplatin. Similarly, both cell lines exhibited sensitivity to β-defensin-2 and cathelicidin-1 peptides; however, the comparative analysis of these peptides on inhibiting both cell lines showed that cathelicidin-1 had a greater inhibitory effect on both cancer cell lines than β-defensin-2. The IC₅₀ values of cathelicidin-1 were 76.11 μg/mL and 65.58 μg/mL for HT-29 and HCT-116, respectively. β-defensin-2 peptide had IC₅₀ values of 569.31 μg/mL and 868.91 μg/mL for HT-29 and HCT-116, respectively. HT-29 cells were more susceptible to β-defensin-2 treatment than HCT-116 cells. For cytotoxicity, both cancer cell lines' viability was reduced following cathelicidin-1 treatment (1.02±0.03 and 1.93±0.45 for HT-29 and HCT-116, respectively). β-defensin-2 lowered the viability of HT-29 cells to 2.01±0.54 and HCT-116 cells to 38.74+1.22. These evaluations suggest that chicken peptides (cathelicidin-1 or defensin-2) may be therapeutic anticancer agents for the treatment of drug-resistant colon cancer.

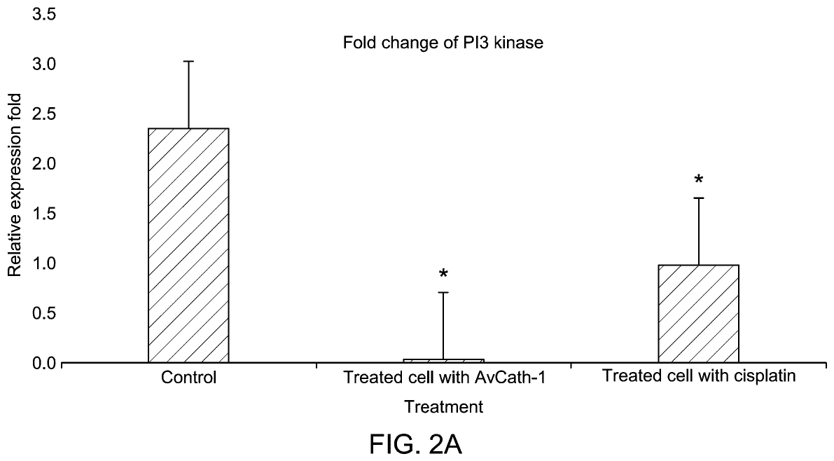

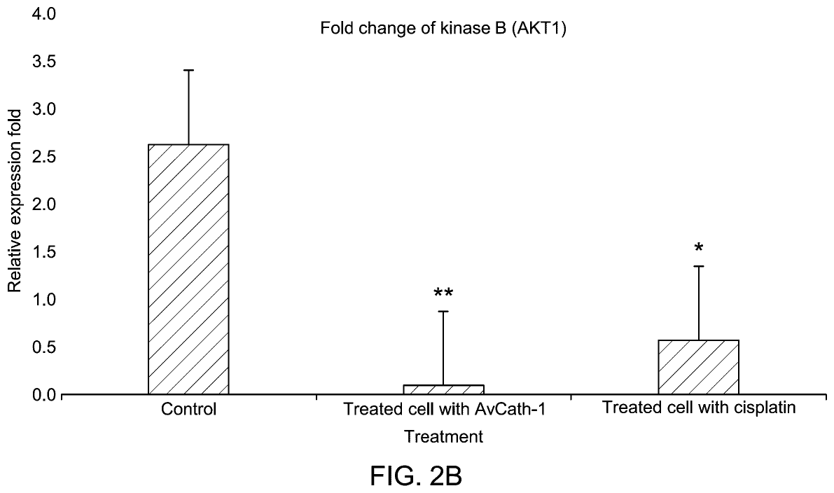

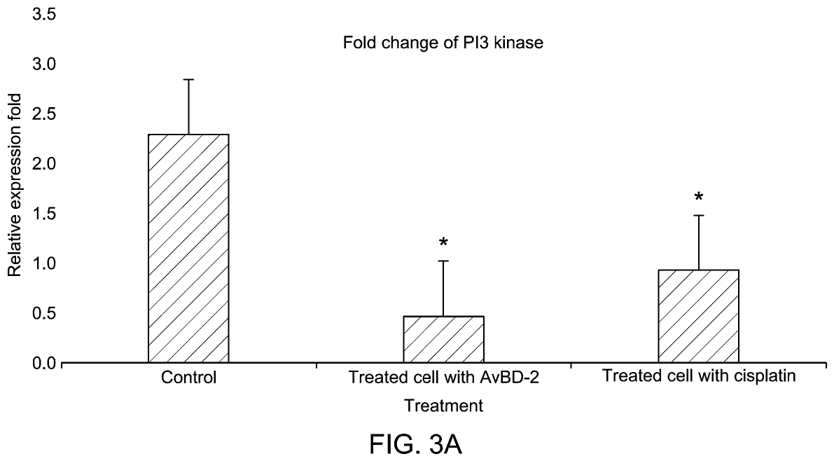

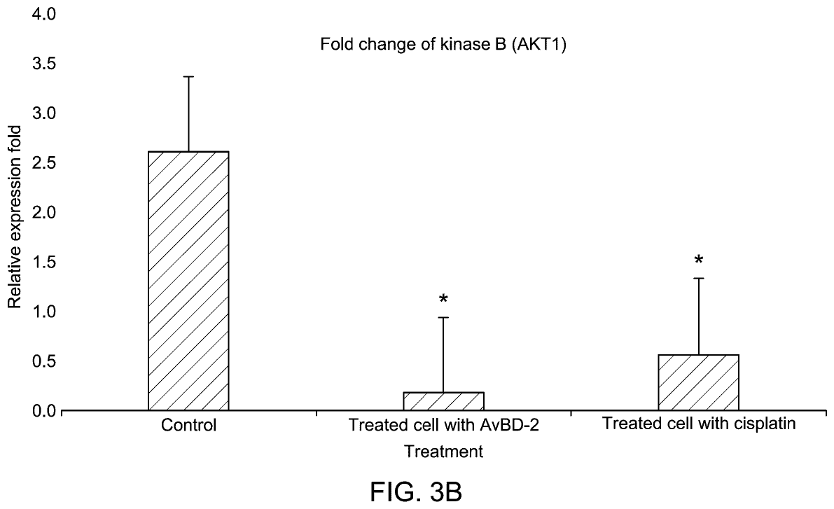

As demonstrated in FIG. 2A & FIG. 2B, the treatment of chicken cathelicidin-1 and cisplatin in the HCT-116 cancer cell line resulted in the alteration of PI3 kinase and Akt1 expression. Genes associated with the PI3K/Akt signal transduction pathway, including PI3K and Akt1, are used in regulating cell growth, survival, and metabolism through signaling cascades. In the untreated cells (HCT-116 controls), a two-fold expression of the PI3K and Akt1 genes was observed; however, cells treated with chicken cathelicidin-1 did not exhibit apparent expression due to the downregulation of these genes. In contrast, cisplatin-treated cells showed a one-fold increase in expression compared to untreated cells. This suggests that the chicken cathelicidin-1 peptide may be an anticancer agent, as it affects the downregulation of PI3K and Akt1 genes in colon cancer cells. FIG. 3A & FIG. 3B demonstrate the treatment of chicken β-defensin-2 peptide and cisplatin in the HCT-116 cancer cell line resulted in the alteration of PI3 kinase and Akt1 expression. In the untreated cells (HCT-116 controls), a two-fold expression of the PI3K and Akt1 genes was observed; however, cells treated with the β-defensin-2 peptide exhibited an apparent expression of less than 0.5 due to the downregulation of these genes. In contrast, cisplatin-treated cells showed a one-fold increase in expression compared to untreated cells. This suggests that the β-defensin-2 peptide may be an anticancer agent, as it affects the downregulation of PI3K and Akt1 genes in colon cancer cells.

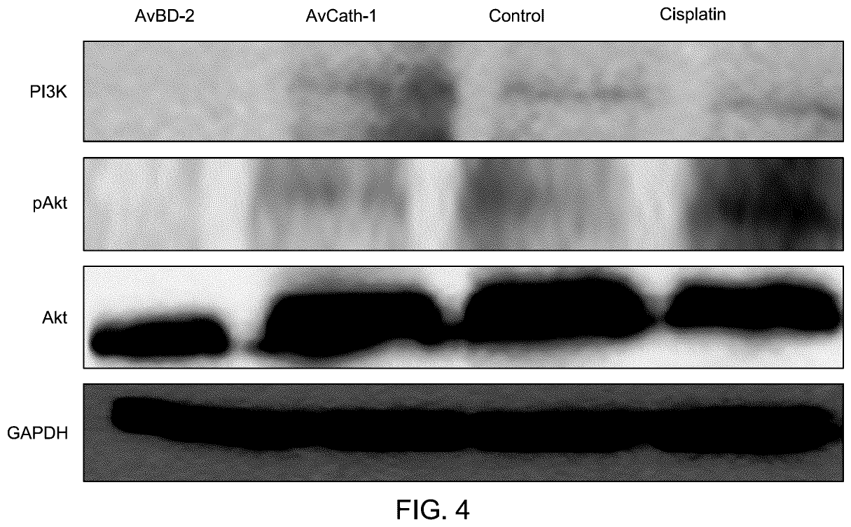

Quantitative PCR was conducted to investigate the expression of the PI3 kinase and Akt1 genes in HCT-116 cells treated with the chicken β-defensin-2 peptide, chicken cathelicidin-1 peptide, and cisplatin. These evaluations revealed that in untreated cells (HCT-116 control), both genes were expressed. In contrast, cells treated with chicken β-defensin-2 peptide showed no apparent expression, suggesting that the peptide directly regulates the downregulation of these genes, as illustrated in FIG. 4.

Similar to other forms of cancer, colon cancer exhibits multidrug resistance to currently available chemotherapeutic regimens. The current disclosure evaluates the levels of total and phosphorylated Akt1 protein, as activation of this signaling pathway is mediated by PI3K. HCT-116 cells were treated with cisplatin, chicken cathelicidin-1, and β-defensin-2, and the expression of phosphorylated Akt and PI3K was decreased compared to the untreated control group. While all three agents reduced protein levels, β-defensin-2 treated cells showed low levels of expression. β-defensin-2 may be used as an anticancer agent as it impacted the downregulation of PI3K/Akt signaling pathway and protein levels.

Anticancer activity of chicken β-defensin-2 and cathelicidin-1 peptides in drug-resistant colon cancer cell lines (HT-29 and HCT-116) is compared to cisplatin. A mechanism of action of cationic peptide interactions with colon cancer cell lines to assess kinase inhibitor activity is determined. Various peptide doses for 48 hours were evaluated to determine their effects on colon cancer cell lines HT-29 and HCT-116. Both cell types were sensitive to β-defensin-2 and cathelicidin-1 peptides, indicating their potential as therapeutic agents. When comparing the IC₅₀ values between the HT-29 and HCT-116 cell lines, cathelicidin-1 displayed the highest inhibition in both cancer cell lines compared to β-defensin-2; however, HT-29 cells were more susceptible to β-defensin-2 treatment than HCT-116 cells. Cathelicidin-1 treatment reduced the viability of both cancer cell lines, while β-defensin-2 reduced the viability of HT-29 cells more than that of HCT-116 cells.

Chicken β-defensin-2 and cathelicidin-1 peptides can indirectly induce cancer cell death. The mechanism of action of these peptides involves the downregulation of certain genes that are used in the growth, proliferation, and development of colon cancer cells. This leads to inhibition of cell growth and division, often resulting in cell death. To elucidate the target signaling pathways of these peptides, involvement of the PI3K/Akt signal transduction network in HCT-116 colon cancer cells after treatment with chicken β-defensin-2 and cathelicidin-1 peptides was investigated. Further, the chicken cathelicidin-1 peptide exhibited downregulation of PI3K and AKT1 gene expression in colon cancer cells as determined by RT-qPCR. Cells treated with chicken β-defensin-2 also displayed less visible expression of these genes. Chicken β-defensin-2 peptide decreased the expression of PI3K and Akt1 genes in colon cancer cells. These peptides had a greater impact on the expression of PI3K and Akt genes than cisplatin in a colon cancer cell line. Cisplatin decreased the expression of PI3K and Akt1 transcripts compared to untreated cells.

Treatment of HCT-116 cells with cisplatin, chicken cathelicidin-1, and β-defensin-2 result in a decrease in phosphorylated Akt and PI3k levels compared to an untreated control group, as determined by western blotting. Protein expression was decreased or undetectable following β-defensin-2 peptide treatment. The β-defensin-2 group exhibited the most prominent influence on the downregulation of PI3K/Akt signaling protein expression compared to other peptide treatments. The effect of cisplatin on PI3K/Akt protein levels was found to be lower in treated cells compared to untreated cells.

Numerous modifications and variations of the present disclosure are possible in light of the above teachings. It is, therefore, to be understood that within the scope of the appended claims, the disclosure may be practiced otherwise than as specifically described herein.

1: A method of treating colon cancer in a sample, comprising:
contacting a peptide with the sample comprising colon cancer cells,
wherein the peptide is a chicken β-defensin-2 peptide,
wherein the peptide inhibits cell growth of the colon cancer cells in the sample through downregulation of a PI3K signaling pathway and an Akt1 signaling pathway.

2: The method of claim 1, wherein the chicken β-defensin-2 peptide has an amino acid sequence at least 90% identical to SEQ ID No. 1.

3: The method of claim 1, wherein the chicken β-defensin-2 peptide has an amino acid sequence at least 95% identical to SEQ ID No. 1.

4: The method of claim 1, wherein the chicken β-defensin-2 peptide has an amino acid sequence at least 99% identical to SEQ ID No. 1.

5: The method of claim 1, wherein the chicken β-defensin-2 peptide has a half-maximal inhibitory concentration of 865 to 875 μg/mL when administered to colorectal cancer cells, HT-116.

6: The method of claim 1, wherein the chicken β-defensin-2 peptide has a half-maximal inhibitory concentration of 565 to 575 μg/mL when administered to colorectal cancer cells, HT-29.

7: The method of claim 1, wherein the colon cancer cells are colorectal cancer cells HT-116, and wherein contacting the colorectal cancer cells HT-116 with the chicken β-defensin-2 peptide at a concentration of 1000 μg/mL reduces a cell viability of the colorectal cancer cells 5 HT-116 by 68 to 74%.

8: The method of claim 1, wherein the colon cancer cells are colorectal cancer cells HT-29, and wherein contacting the colorectal cancer cells with the chicken β-defensin-2 peptide at a concentration of 1000 μg/mL reduces a cell viability of the colorectal cancer cells HT-29 by 97 to 99%.

9: The method of claim 1, wherein the colon cancer cells are colorectal cancer cells HCT-116, and wherein contacting the colorectal cancer cells with the chicken β-defensin-2 peptide has a relative expression fold change of a PI3K gene of the colorectal cancer cells HCT-116 of 0.4 to 0.6 normalized to β-actin.

10: The method of claim 1, wherein the colon cancer cells are colorectal cancer cells HCT-116, and wherein contacting the colorectal cancer cells with the chicken β-defensin-2 peptide has a relative expression fold change of an Akt1 gene of the colorectal cancer cells HT-29 of 0.1 to 0.3 normalized to β-actin.

11: The method of claim 1, wherein the peptide is a chicken cathelicidin-1 peptide.

12: The method of claim 11, wherein the chicken cathelicidin-1 peptide has an amino acid sequence at least 90% identical to SEQ ID No. 2.

13: The method of claim 11, wherein the chicken cathelicidin-1 peptide has an amino acid sequence at least 95% identical to SEQ ID No. 2.

14: The method of claim 11, wherein the chicken cathelicidin-1 peptide has an amino acid sequence at least 99% identical to SEQ ID No. 2.

15: The method of claim 11, wherein chicken cathelicidin-1 peptide has a half-maximal inhibitory concentration of 64 to 68 μg/mL when administered to colorectal cancer cells, HCT-116.

16: The method of claim 11, wherein chicken cathelicidin-1 peptide has a half-maximal inhibitory concentration of 74 to 78 μg/mL when administered to colorectal cancer cells, HCT-29.

17: The method of claim 11, wherein the colon cancer cells are colorectal cancer cells HT-116, and wherein contacting the colorectal cancer cells HT-116 with the chicken cathelicidin-1 peptide at a concentration of 1000 μg/mL reduces a cell viability of the colorectal cancer cells HT-116 by 95 to 99%.

18: The method of claim 1, wherein the colon cancer cells are colorectal cancer cells HT-29, and wherein contacting the colorectal cancer cells with the chicken β-defensin-2 peptide at a concentration of 1000 μg/mL reduces a cell viability of the colorectal cancer cells HT-29 by 98.5 to 99.5%.

19: The method of claim 1, wherein the colon cancer cells are colorectal cancer cells HCT-116, and wherein contacting the colorectal cancer cells with the chicken cathelicidin-1 peptide has a relative expression fold change of a PI3K gene of the colorectal cancer cells HCT-116 of 0.01 to 0.1 normalized to β-actin.

20: The method of claim 1, wherein the colon cancer cells are colorectal cancer cells HCT-116, and wherein contacting the colorectal cancer cells with the chicken cathelicidin-1 peptide has a relative expression fold change of an Akt1 gene of the colorectal cancer cells HT-29 of 0.05 to 0.3 normalized to β-actin.
